(19) United States
(12) Patent Application Publication
(10) Pub. No.: US 2013/0018091 A1
(43) Pub. Date: Jan. 17, 2013

(54) TREATMENT OF ALLODYNIA AND HYPERALGESIA

(71) Applicants: Peter Blower, London (GB); Argeris Karabelas, London (GB)

(72) Inventors: Peter Blower, London (GB); Argeris Karabelas, London (GB)

(21) Appl. No.: 13/513,249

(22) PCT Filed: Dec. 3, 2010
(86) PCT No.: PCT/GB2010/052021

§ 371 Date: Oct. 2, 2012

Related U.S. Application Data
(60) Provisional application No. 61/226,205, filed on Jul. 16, 2009.

(51) Int. Cl. A61K 31/353; A61P 29/00; A61P 25/00; C07D 311/68

(52) U.S. Cl. 514/456; 549/399

(57) ABSTRACT

The present invention relates to the treatment or prevention of allodynia and/or hyperalgesia, and to tonabersator an analogueof formula (1), and compositions comprising tonabersat or an analogue of formula (1) for use in said treatments.

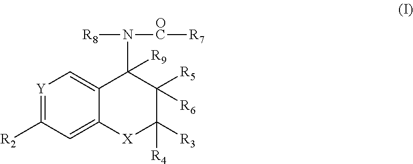

The present invention relates to tonabersat and its analogues and compositions comprising tonabersat or its analogues for use in the treatment or prevention of allodynia and hyperalgesia.

International patent application WO 95/34545 discloses a series of specific named compounds, including tonabersat, otherwise known as cis-6-acetyl-4-(S)-(3-chloro-4-fluorobenzoylamino)-3,4-dihydro-2,2-dimethyl-2H-1-benzopyran-3-(S)-ol, which is a member of the class of drugs called neuronal gap junction blockers, and which is currently being investigated for a range of conditions including migraine, epilepsy, depression and other neurological conditions.

U.S. Pat. No. 5,948,811 (incorporated herein by way of reference) describes a class of compounds (‘the analogues of formula I’) which may be used for the prophylaxis and treatment of disorders within the central and peripheral nervous system, including migraine, psychosis, epilepsy and other neurological conditions.

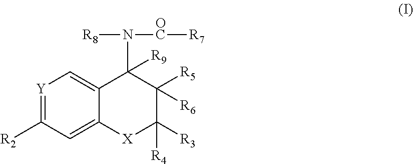

Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—,
—CO—, —CH₂—, CH(OH), SO₂, SO, CH₂—O, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇ alkanoyl; one of R₃ and R₄ is hydrogen or C₁₋₄ alkyl and the other is C₁₋₄ alkyl, CF₃ or CH₂X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C.sub.1-6 alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆ alkyl.

Allodynia is generally defined as a pain due to a stimulus which does not normally provoke pain. It usually involves an unpleasant, abnormal sensation or dysesthesia. There are two different kinds of allodynia; mechanical and thermal. Static mechanical allodynia is pain in response to light touch or pressure and dynamic mechanical allodynia is pain in response to brushing. Thermal allodynia refers to pain from normally mild skin temperatures in the affected area. Allodynia is a clinical feature of many painful conditions and is associated with neural disorders, neuro-hypersensitive disorders, inflammatory conditions, nerve compression and/or entrapment and trauma, including postoperative trauma and phantom limb pain. Allodynia differs from referred pain.

Hyperalgesia is an extreme reaction and hypersensitivity to painful stimuli which may be caused by damage to nociceptors or peripheral nerves. Hyperalgesia can be experienced in focal or diffuse form. The focal form is typically associated with injury and is divided into two subtypes; primary hyperalgesia and secondary hyperalgesia. Primary hyperalgesia describes pain sensitivity that occurs directly in the damaged tissues. Secondary hyperalgesia describes pain sensitivity that occurs in surrounding undamaged tissues. Hyperalgesia is induced by platelet-activating factor (PAF) in patients with inflammatory or allergic conditions. It occurs via immune cells interacting with the peripheral nervous system and releasing pain-producing chemicals. Patients on long-term or high-dose opioid medication for the treatment of chronic pain may experience hyperalgesia.

Allodynia and hyperalgesia as with other pain associated with nerve damage such as neuropathic pain may respond to standard treatment using various drugs such as selective serotonin re-uptake Inhibitors, tricyclic antidepressants, non-steroidal anti-inflammatory drugs, glucocorticoids, gabapentin or pregabalin, NMDA antagonists or atypical opioids. Compounds that treat allodynia include Lidocaine, Alfentanyl, Adenosine, Ketamine, Glycine antagonist and Venlafaxine. However, as with other forms of nerve dysfunction associated pain, treatment of allodynia and/or hyperalgesia is clinically challenging and not all patients respond, hence there exists a critical need for safe, alternative and improved methods and compositions for the treatment or prevention of allodynia and/or hyperalgesia.

In a first aspect, the present invention provides for tonabersat, or an analogue of formula I,

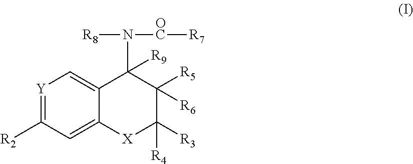

Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—, —CO—, —CH₂—, CH(OH), SO₂, SO, CH₂—O, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇, alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇, alkanoyl; one of R₃ and R₄ is hydrogen or C₁₋₄ alkyl and the other is C₁₋₄ alkyl, CF₃ or CH₂X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C.sub.1-6 alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆ alkyl or a pharmaceutically acceptable composition thereof, for use in the treatment or prevention of allodynia and/or hyperalgesia.

For therapeutic administration according to the present invention, tonabersat or an analogue of formula I, is most preferably employed in the form of its free base, but may also be used in the form of a pharmaceutically acceptable salt, preferably the hydrochloride salt. Alternative salts with pharmaceutically acceptable acids may also be utilised in prophylactic and/or therapeutic administration, for example salts derived from acids including, but not limited to, hydrobromic acid, phosphoric acid, acetic acid, fumaric acid, maleic acid, salicylic acid, citric acid, oxalic acid, lactic acid, malic acid, methanesulphonic acid and p-toluene sulphonic acid.

All references to tonabersat or an analogue of formula I, herein includes all pharmaceutically acceptable salts, and all solvates thereof.

Also included within the scope of the present invention are polymorphs, solvates and radiolabelled derivatives of tonabersat or an analogue of formula I, and pharmaceutically acceptable compositions thereof. References to tonabersat or an analogue of formula I, include such polymorphs, solvates and radiolabelled derivatives thereof.

For prophylactic and/or therapeutic administration according to the invention, tonabersat or an analogue of formula I, may be administered in pure form, but will preferably be formulated into any suitable pharmaceutically acceptable and effective composition which provides effective levels of the active ingredient in the body.

Accordingly, the present invention provides a method for the treatment or prevention of allodynia and/or hyperalgesia, comprising administering to a patient in need thereof a pharmaceutically effective amount of tonabersat or an analogue of formula I, or a pharmaceutically acceptable composition thereof.

In a further aspect, the present invention therefore provides for the tonabersat or an analogue of formula I, or a pharmaceutically acceptable composition thereof, for use in the treatment or prevention of allodynia and/or hyperalgesia associated with neural disorders, neuro-hypersensitive disorders and/or one or more associated symptoms.

Accordingly, the present invention provides a method for the treatment or prevention of allodynia and/or hyperalgesia associated with neural disorders, neuro-hypersensitive disorders, comprising administering to a patient in need thereof a pharmaceutically effective amount of tonabersat or an analogue of formula I, or a pharmaceutically acceptable composition thereof.

In further aspect, the present invention provides for tonabersat or an analogue of formula I, or a pharmaceutically acceptable composition thereof, for use in the treatment or prevention of allodynia and/or hyperalgesia associated with inflammatory disorders and/or one or more associated symptoms.

In further aspect, the present invention provides for tonabersat or an analogue of formula I, or a pharmaceutically acceptable composition thereof, for use in the treatment or prevention of glossodynia, or burning mouth syndrome. Burning mouth syndrome is a burning sensation of the tongue or mouth, which may be related to problems with taste and/or sensory nerves of the peripheral or central nervous system.

Accordingly, the present invention provides a method for the treatment or prevention of allodynia and/or hyperalgesia associated with inflammatory disorders, comprising administering to a patient in need thereof a pharmaceutically effective amount of tonabersat or an analogue of formula I, or a pharmaceutically acceptable composition thereof.

In a further aspect, the present invention provides for tonabersat or an analogue of formula I, or a pharmaceutically acceptable composition thereof, for use in the treatment or prevention of hyperalgesia associated with nerve compression, nerve entrapment, trauma more particularly post operative trauma, phantom limb pain and/or one or more associated symptoms.

Accordingly, the present invention provides a method for the treatment or prevention of allodynia and/or hyperalgesia associated with nerve compression, nerve entrapment, trauma, more particularly post operative trauma, phantom limb pain comprising administering to a patient in need thereof a pharmaceutically effective amount of tonabersat or an analogue of formula I, or a pharmaceutically acceptable composition thereof.

The invention further comprises the use of tonabersat in the manufacture of a medicament for the treatment of any one or more of the uses listed above.

All treatments may be acute or prophylactic. For acute treatment, a rapid onset of action is preferred, and therefore, drugs that reach maximum plasma concentrations shortly after administration would be most beneficial. Accordingly, compositions providing rapid drug-release and/or dissolution are preferred.

Tonabersat or an analogue of formula I, may be delivered alone, but will generally be delivered in the form of a pharmaceutically acceptable composition thereof, which comprises tonabersat or an analogue of formula I, and one or more pharmaceutically acceptable diluents or carriers selected with regard to the intended route of administration.

Treatment with tonabersat or an analogue of formula I, or a pharmaceutically acceptable composition thereof, may be conducted at a unit dose of between 1 to 1000 mg, suitably 1 to 500 mg, for example an amount in the range of from 2 to 400 mg such as 2, 5, 10, 20, 30, 40, 50, 80, 100, 200, 300 and 400 mg of the active compound.

Unit doses will normally be administered once or more than once per day, for example 1, 2, 3, 4, 5 or 6 times a day, more usually 1 to 4 times a day, such that the total daily dose is normally in the range, for a 70 kg adult of 1 to 1000 mg, for example 1 to 500 mg, that is in the range of approximately 0.01 to 15 mg/kg/day, more usually 0.1 to 6 mg/kg/day, for example 1 to 6 mg/kg/day.

Preferably, the tonabersat or an analogue of formula I, or a pharmaceutically acceptable salt thereof, is administered to the patient at dose ranges of approximately 0.01 to 15 mg/kg/day, more usually 0.1 to 6 mg/kg/day, for example 1 to 6 mg/kg/day.

It is preferred that tonabersat or an analogue of formula I, is administered in the form of a pharmaceutical composition, such as a composition for oral, including sub-lingual, intranasal, rectal, topical, parenteral (especially intravenous), ocular or aural administration.

Pharmaceutical compositions suitable for the delivery of tonabersat or an analogue of formula I, and methods for their preparation will be readily apparent to those skilled in the art. Such compositions and methods for their preparation may be found, for example, in Remington's Pharmaceutical Sciences, 19th Edition (Mack Publishing Company, 1995).

Compositions suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations. Liquid formulations include suspensions, solutions, syrups and elixirs. Liquid formulations may also be prepared by the reconstitution of a solid, for example, from a sachet.

Orally administrable compositions may be in the form of oral solid compositions, such as tablets, capsules, pastilles, pellets, pills, lozenges powders and granules. The composition may be in solid form which melts on contact with the tongue of the patient, for example in the form of disintegrating tablets sold under the trade name ZYDIS®. Shaped oral compositions are preferred, since they are more convenient for general use.

Solid forms for oral administration are usually presented in a unit dose, and contain conventional additives such as adjuvants, binding agents, diluents, disintegrants, dispersing agents, excipients, fillers, tabletting agents, lubricants, colorants, flavourings, desiccants, humectants, and wetting agents.

Pills, pellets and tablets may be coated according to well known methods in the art. Oral solid formulations also include conventional sustained release formulations, such as tablets or granules having an enteric coating.

Suitable fillers include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate.

Solid oral compositions are prepared by admixture, and may be prepared by conventional methods of blending, filling, tabletting or the like. Repeated blending operations may be used to distribute the active agent throughout those compositions employing large quantities of fillers. Such operations are, of course, conventional in the art.

They may also be in the form of oral fluid preparations, including liquid preparations, such as aqueous or oily blends, mixtures, suspensions, solutions, emulsions, syrups, tinctures and elixirs, and gel preparations.

They may also be presented as dilutable fluid concentrates or dry product reconstitutable powders for dilution or reconstitution with water or other suitable vehicle before use.

Oral fluid preparations, including gels and liquid preparations may contain conventional additives such as: suspending agents, for example sorbitol, syrup, methyl cellulose, gelatin, hydroxyethylcellulose, carboxymethyl cellulose, aluminium stearate gel or hydrogenated edible fats; emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid; and, if desired, conventional flavouring or colouring agents.

Compositions for oral administration may be formulated to be immediate and/or modified release. Modified release formulations include delayed-, sustained-, pulsed-, controlled-, targeted and programmed release.

Compositions suitable for parenteral administration include injectable and infusible aqueous or oily blends, mixtures, suspensions, solutions, emulsions and low-viscosity gel preparations. Compositions for parenteral administration may be formulated to be immediate and/or modified release. Modified release formulations include delayed-, sustained-, pulsed-, controlled-, targeted and programmed release.

The parenteral compositions for use in the invention may be prepared as long acting depot preparations. Such formulations may be administered by intramuscular injection. Thus, for example, the composition of the invention may be formulated with suitable polymeric or hydrophobic materials (for example as an emulsion in a pharmaceutically acceptable oil) or ion exchange resins, or as sparingly soluble derivatives, for example, as a sparingly soluble salt.

Advantageously, adjuvants such as a local anaesthetic, preservatives and buffering agents are also dissolved, emulsified or suspended in the vehicle.

Such compositions are prepared by admixture of the compound and a solvent or vehicle. The compound, depending on the vehicle and the concentration, can be emulsified, suspended or dissolved. Parenteral compositions are normally prepared by with the compound and a vehicle which is sterile, and/or the composition is sterilised, before filling into a suitable vial or ampoule and sealing.

To enhance the stability, the composition may also be presented as a dry product reconstitutable powder for reconstitution with water or other suitable vehicle before use. A fluid composition can be frozen after filling into the vial and freeze-dried under vacuum.

Tonabersat or an analogue of formula I, may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™ Bioject™, etc.) injection.

Formulations for topical administration may be formulated to be immediate and/or modified release. Modified release formulations include delayed-, sustained-, pulsed-, controlled-, targeted and programmed release.

Compositions for use in surgical wounds may be prepared as long acting depot preparations. Such formulations may be administered by implantation (for example subcutaneously or intramuscularly) or by intramuscular injection. Thus, for example, the composition of the invention may be formulated with suitable polymeric or hydrophobic materials (for example as an emulsion in an acceptable oil) or ion exchange resins, or as sparingly soluble derivatives, for example, as a sparingly soluble salt.

It is more preferred that tonabersat or an analogue of formula (I) is administered in the form of a unit-dose composition for administration into a temporary or permanent human or other animal bodily orifice, such as the trachea, nostril, nasal passage, rectum, udder duct, urethra or vagina, or a surgical would, e.g. an incision, or any device inserted in such a temporary or permanent orifice, such as a catheter, trochar, cannula, endotracheal or other endoscopic tube or an ostomy tube, e.g. a tracheostomy or colostomy tube. Intranasal administration is greatly preferred

Intranasally mucosal administrable compositions may be in the form of intranasal mucosal solid compositions, such as powders and granules. They may also be in the form of intranasal mucosal fluid preparations, including liquid preparations, such as aqueous or oily blends, mixtures, suspensions, solutions, emulsions and elixirs, and gel preparations. They may also be presented as dilutable fluid concentrates or dry product reconstitutable powders for dilution or reconstitution with water or other suitable vehicle before use.

Tonabersat or an analogue of formula I, may be administered intranasally or by inhalation, typically in the form of a dry powder from a dry powder inhaler (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

Solid forms for intranasal mucosal administration are usually presented in a unit dose, and contain conventional additives, such as adjuvants, diluents, dispersing agents, excipients, colorants, desiccants, humectants, and wetting agents.

Powders and granules may be coated according to well known methods in the art. Intranasal mucosal solid formulations also include conventional sustained release formulations, such as powders or granules having a resistant coating.

Suitable excipients include cellulose, mannitol, lactose, chitosan, pectin, cellulose derivatives such as hydroxypropylmethylcellulose, methyl cellulose, hydroxyethylcellulose, carboxymethyl cellulose, polyoxamers, such as poly(ethylene oxides), gelatin, polyvinylpyrrolidone and starch. Suitable pharmaceutically acceptable wetting agents include sodium lauryl sulphate.

Solid intranasal mucosal compositions are prepared by admixture, and may be prepared by conventional methods of blending or the like. Repeated blending operations may be used to distribute the active agent throughout those compositions employing large quantities of excipients. Such operations are, of course, conventional in the art.

Intranasal mucosal fluid preparations, including gels and liquid preparations may contain conventional additives such as suspending agents, for example sorbitol, methyl cellulose, gelatin, hydroxyethylcellulose, carboxymethyl cellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional colouring agents.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise an analogue of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Tonabersat or an analogue of formula I, may also be administered rectally or vaginally, for example, in the form of a suppository, pessary, or enema.

Tonabersat or an analogue of formula I, may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

The compositions of tonabersat or an analogue of formula I, may also be in the form of fast-dispersing dosage forms such as those described in Expert Opinion in Therapeutic Patents, 11 (6), 981-986, by Liang and Chen (2001) and Verma RK et.al. Current Status of Drug Delivery Technologies and Future Directions, Pharmaceutical Technology On-Line, 2001, 25(2), 1-14. Such dosage forms are also known as oral fast-dissolving, rapid-dissolve, rapid-melt, mouth-dissolving and fast-disintegrating tablet. The composition may be in solid form which melts on contact with the tongue of the patient, for example in the form of disintegrating tablets sold under the trade name ZYDIS® (RP Scherer, UK). Alternatively, the composition may be in the form of the EFVDAS (effervescent drug absorption system, Elan Corporation), Fast Melt (highly porous microfine matrix tablet, Elan Corporation), Flashdose (floss matrix utilising shearform technology, (Fuisz Technologies, USA), Flashtab (orodispersible multiparticulate tablet, Prographarm, France), Multiflash (fast disintegrating multi-unit, multiparticulate tablet, Prographarm), Orasolv (effervescent dispersed microcapsule tablet, Cima Labs Inc, USA), Wowtab tablets (Yamanouchi Pharma Technologies, USA), LYOC (freeze dried fast dispersing tablets, Farmalyoc, France) or Quicksolve (freeze dried fast dispersing tablets, Janssen Pharamceutica, USA).

Other suitable formulation technologies may include INDAS (insoluble drug absorption system, Elan Corporation), which utilises a stabilised amorphous form of the drug with enhanced solubility, NanoCrystal technology (Elan Corporation), which utilises nanoparticles of the drug, typically having a particle size of less than 400 nm in diameter, or SoftGel (RP Scherer), which utilises a soft gelatin capsule formulation.

The formulation technologies described herein may advantageously provide more rapid drug dissolution and absorption. For those compositions that disintegrate in the oral cavity, such as beneath the tongue, the rate of absorption may be increased and first-pass metabolism effects reduced.

As is common practice, the compositions will usually be accompanied by written or printed directions for use in the medical treatment concerned.

The compositions for use in the invention may contain from 0.1% to 99% by weight, preferably from 1%-60% by weight, of the active material, depending on the method of administration.

The following are given by way of example only to illustrate and aid understanding of the invention:

Studies with tonabersat have employed a number of different formulations including:
Direct compression tablets 0.05, 1.0, 10 and 25 mg with tablet core weight 250 mg
Direct compression tablets 15, 25, 40 and 80 mg with tablet core weight 400 mg
Direct compression tablets 20 mg with core weight 400 mg
Nanoparticulate tablets 10, 20 and 40 mg with tablet core weight 400 mg

The direct compression tablets utilise micronized drug substance whilst the nanoparticulate tablets were direct compression tablets utilising wet bed milled spray dried nanoparticulate drug substance. Clinical trials have been conducted utilising 10, 20, 30, 40, 60 and 80 mg round white uncoated direct compression tablets with a core weight of 400 mg with the following unit composition (20 mg tablet only presented; all other strengths differ only in tonabersat and lactose content):

A representative formulation suitable for use in the present invention is detailed in Table 1.

TABLE 1

| | 20 mg Tablet |
|---|---|
| Ingredient | Quantity (mg) |
| Tonabersat | 20.0 |
| Lactose | 330.0 |
| Microcrystalline Cellulose | 20.0 |
| Sodium Starch Glycollate, | 24.0 |
| Type A | |
| Colloidal Silicon Dioxide | 2.0 |
| Magnesium Stearate | 4.0 |
| Total Weight | 400.0 |

### EXPERIMENTAL METHODS

### Example 1

### Formalin-Induced Hyperalgesia and Inflammation

### Aim

To determine the effect of tonabersat on chronic allodynia and/or hyperalgesia models.

### Method

An injection of 0.5% formalin is made into the planter region of a CF1 mouse (obtained from Charles River, Inc.) right hind paw. This elicits a distinct biphasic behavioral profile characterized by the mouse licking the affected paw. Immediately following the injection the mouse licks the paw for about 10 minutes. This is phase 1 (acute) and is followed by a brief latent period where there is little behavioral activity. A more prolonged period of about 20 to 30 minutes of paw licking ensues which constitutes phase 2 (inflammatory).

Prior to the administration of the test drug or vehicle each mouse undergoes a 15-minute conditioning period in one of several 6″ tall plexiglass observation tubes (4″ diameter) that are placed in front of a mirror. Following the conditioning period, doses of tonabersat will be administered p.o. and the mouse returned to its home tube. At a time determined to correlate with peak plasma levels (1 hour), formalin will be injected sub-dermally (20 ul; 27 gauge needle) into the plantar surface of the right hind foot. The bevel of the needle is placed facing down toward the skin surface. Following injection of the formalin each animal is observed for the first 2 minutes of each 5 minute epoch for a total of 45 minutes. The cumulative length of licking for each 2 minute time period is measured. An animal receiving the requisite volume of vehicle is alternated with each mouse given the test drug. Animals are euthanized following the conclusion of the experiment. An n of 8 mice per group will be employed in this study.

Area under the curve (AUC) determination is made using the GraphPad Prism Version 3.03. Total AUC is calculated for both the test and control groups for both the acute and inflammatory phases. The AUC for individual animals for each phase is also calculated and converted to percentage of total AUC of control. The average percentages and SEM for both the drug treated and control are calculated and tested for significant difference.

### Results

Tonabersat—Test data from formalin-induced nociception model in mice (n=8/group). Effect on duration of licking during the two phases (acute and inflammatory) of the test.

| | | | Drug- | % | | |
|---|---|---|---|---|---|---|
| Dose | | Control | treated | Inhibition | | Statistical |
| mg/kg | Test | AUC | AUC | of Control | SEM | Analysis |
| 45.0 | Acute | 185.8 | 116.4 | 37.3 | +/−14.7 | p = >0.05 |
| | Phase | | | | | |
| 45.0 | Inflam- | 446.7 | 135.8 | 69.6 | +/−11.6 | p = <0.05 |
| | matory | | | | | |
| | Phase | | | | | |

### Example 2

### Sciatic Ligation-Induced Peripheral Nerve Sensitization

### Aim

To determine the effect of tonabersat on a sciatic nerve ligation model. The sciatic nerve ligation model is the ‘gold standard’ model for allodynia and/or hyperalgesia. Both gabapentin and pregabalin are active in these models.

### Method

Following a 10-day post-operative period after partial ligation of the sciatic nerve as described by Seltzer et al, each of the operated Sprague-Dawley rats (obtained from Charles River, Inc.) will be tested for the development of consistent, mechanical allodynia (pain response to a non-noxious stimulus). Animals are placed in a bottomless plexiglass box placed on a stainless steel platform with ¼″ holes. After at least a 30-min conditioning period in which rats are allowed to sit quietly in the cages on the platform, a baseline mechanical sensitivity is determined which represents the pre-drug 50% paw withdrawal threshold. This procedure is done by applying a series of calibrated Von Frey monofilament fibers perpendicularly to the plantar surface of each hind paw in between the pads or further back toward the heel. The 50% threshold for foot withdrawal is determined by using the “Up and Down” step procedure. That is, after a positive response (X=withdrawal of the foot) is noted, a weaker fiber is applied until a failure to withdraw the paw (O=no withdrawal) is noted. This is the estimated threshold value. Once an estimate of the individual rat's threshold has been obtained, the next highest/stiffer/thicker fiber is applied and so forth. This is repeated for 5 steps. If the pressure of the largest diameter fiber raises the foot without a withdrawal, it is considered as “no response.” and recorded as >300 g on the data sheet.

Following the measurement of the initial predrug responses, the rats will be given an p.o. injection of tonabersat (n=8 rats per dose level). The mechanical threshold will then be assessed at 1, 2, 4, 6, 8, and 24 hrs post-injection to determine the duration of action of the test compound and the TPE in this model of mechanical allodynia. The animals may be used again after a sufficient time has elapsed for wash-out of the drug, typically 7 days.

The withdrawal threshold for each animal determined at each time point is computed using the “xoxox” step procedure originally developed by Dixon (1980). As described above, the fiber diameters and the pattern of the response are recorded once the threshold has been estimated (the point where the first positive response (x) is followed by the first negative (O) response). According to Chaplan, the ideal response pattern is thus “xoxox”. For each animal the pattern of the response and the final fiber tested (Xf) is entered into an Excel macro that calculates the 50% paw withdrawal threshold using the formula:

50% g threshold=10̂(X_{f}+□□)/10,000

Where X_{f} is the value in log units of the final von frey hair tested, □ is the tabular value for the pattern of positive/negative responses observed (Appendix provided in Chaplan modified from Dixon, W. J. (1980) Ann Rev. Pharmacol Toxicol, 20:441-462, and □ is the mean difference in log units between stimuli.

Using the 50% paw withdrawal thresholds determined for each animal, the mean value±S.E.M. of 50% paw withdrawal threshold is then calculated for each rat at each time point. When these individual values are divided by the average for the pre-drug assessment, a percent change relative to the predrug mean (percent control value) is determined for each rat. Using the Grubbs Outlier test (Graphpad Statistical Software), statistical outliers are excluded only when individual responses are found to be >3 standard deviations from the group's mean. The percent change is then averaged for the remaining group at each time point. The data are then tested for significant difference from the predrug value using DOS-based PCS software. The 50% paw withdrawal thresholds±SEM is reported as significant (*) if p<0.05.

### Results

Tonabersat—Test data from chronic sciatic nerve ligation model in rats (n=8/group). Effect on threshold for foot withdrawal (g).

| | Time of peak | | | Statistical |
|---|---|---|---|---|
| Dose mg/kg | effect [h] | % Control | S.E.M. | Analysis |
| 30.0 | 6.0 | 237.0 | +/−52.0 | p = <0.05 |

### Example 3

### Rat Pen-Orbital Von Frey Sensitivity Model

### Aim

To show the effect of tonabersat (10 mg/kg i.p.) on morning pen-orbital von Frey sensitivity threshold in rats following chronic sensitization induced by daily application of an “inflammatory mediator soup” (IS) to the surface of the dura.

### Results

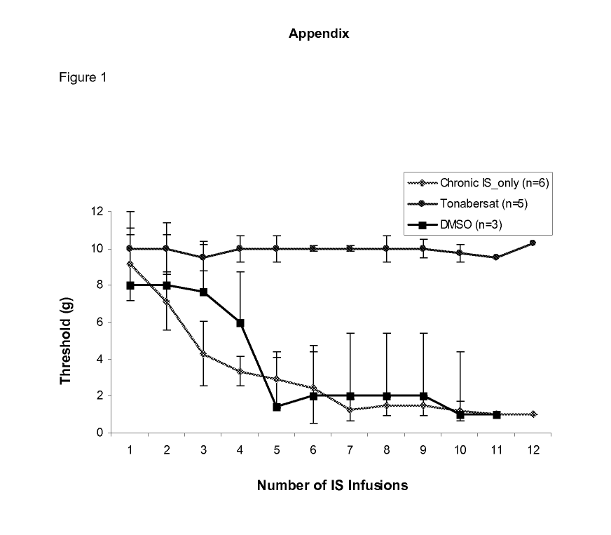

See results graph (FIG. 1) in appendix.

1. Tonabersat or an analogue of formula 1 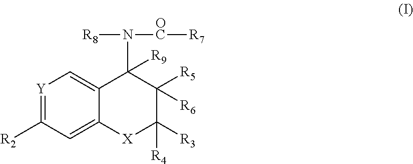
Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—, —CO—, —CH₂ CH(OH), SO₂, SO, CH₂—O, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsuiphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇ alkanoyl; one of R₃ and R₄ is hydrogen or C₁₋₄ alkyl and the other is C₁₋₄ alkyl, CF₃ or CH₂X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, Cl₁₋₆ alkyl, formyl, C₁₋₆ alkanoyl, aroyl or aryl- C₁₋₆ alkyl and R₁₀ is hydrogen, C₁₋₆ alkyl, C₁₋₆ alkoxy, mono or di C. sub.1 -6 alkyl amino, amino, amino-C.sub.1 -6 alkyl, hydroxy- C₁₋₆ alkyl, halo-C₁₋₆ alkyl, C₁₋₆ acyloxy- C₁₋₆ alkyl, C₁₋₆ alkoxycarbonyl- C₁₋₆-alkyl, aryl or heteroaryl; the R₈—N—CO—R₇ group being cis to the R₅ group; and X is oxygen or NR₁₂ where R₁₂ is hydrogen or C₁₋₆ alkyl or a pharmaceutically acceptable composition thereof, for use in the treatment of allodynia and/or hyperalgesia.

2. Tonabersat or an analogue of formula I as defined in claim 1, in the manufacture of a medicament for use in the treatment of allodynia and/or hyperalgesia.

3. A method for the treatment or prevention of allodynia and/or hyperalgesia comprising administering to a patient in need thereof a pharmaceutically effective amount of tonabersat or an analogue of formula I as defined in claim 1.

4. A method of claim 3, wherein the allodynia and/or hyperalgesia is associated with neural disorders.

5. A method of claim 3, wherein the allodynia and/or hyperalgesia is associated neuro-hypersensitive disorders.

6. A method of claim 3, wherein the allodynia and/or hyperalgesia is associated inflammatory conditions.

7. A method of claim 3, wherein the allodynia and/or hyperalgesia is associated with nerve compression and/or entrapment.

8. A method of claim 3, wherein the allodynia and/or hyperalgesia is associated trauma.

9. A method of claim 3, wherein the allodynia and/or hyperalgesia is associated postoperative trauma.

10. A method of claim 3, wherein the allodynia acid/or hyperalgesia is associated phantom limb pain.

11. A method of claim 3, wherein the allodynia and/or hyperalgesia is burning mouth syndrome.
